(19) United States
(12) Patent Application Publication
(10) Pub. No.: US 2024/0180800 A1
(43) Pub. Date: Jun. 6, 2024

(54) Oral Care Compositions Containing Stannous Ion Source

(71) Applicant: Colgate-Palmolive Company, New York, NY (US)

(72) Inventors: Tatiana BRINZARI, Franklin Park, NJ (US); Chi-Yuan CHENG, Hillsborough, NJ (US); Zhigang HAO, Bridgewater, NJ (US); Long PAN, Somerset, NJ (US); Cristina CASTRO, Piscataway, NJ (US); Robert D'AMBROGIO, Prinveton, NJ (US); Jean DENIS, Union, NJ (US)

(73) Assignee: Colgate-Palmolive Company, New York, NY (US)

(21) Appl. No.: 18/529,347

(22) Filed: Dec. 5, 2023

Related U.S. Application Data
(60) Provisional application No. 63/430,229, filed on Dec. 5, 2022.
(60) Provisional application No. 63/430,230, filed on Dec. 5, 2022.

(51) Int. Cl. A61K 8/21; A61K 8/24; A61K 8/27; A61K 8/34; A61K 8/55; A61K 8/67; A61Q 11/00

(52) U.S. Cl. CPC A61K 8/21; A61K 8/24; A61K 8/27; A61K 8/347; A61K 8/55; A61K 8/676; A61Q 11/00; A61K 2800/522

(57) ABSTRACT

Oral care compositions comprising a stannous ion source, tetrasodium pyrophosphate and an antioxidant, as well as to methods of using these compositions are disclosed herein. In some embodiments, the antioxidant is selected from ascorbyl phosphate and ascorbate.

### CROSS-REFERENCE TO RELATED APPLICATIONS

This application claims the benefit of priority from U.S. Provisional Application No. 63/430,229, filed Dec. 5, 2022, and from U.S. Provisional Application No. 63/430,230, titled Oral Care Compositions Containing Stannous Ion Source and filed Dec. 5, 2022, the contents for both of which are hereby incorporated herein in their entireties, for all purposes.

### BACKGROUND

Stannous fluoride (SnF₂) is well known for use in clinical dentistry with a history of therapeutic benefits dating back to the early 1950s. It has been reported to be an effective agent for treating various oral conditions and diseases including plaque, gingivitis, sensitivity, enamel decalcification, and periodontitis, among others. Stannous fluoride is widely used in commercial toothpastes and mouthwashes as an active ingredient delivering anticavity and antigingivitis benefits. However, it is well known that stannous fluoride is sensitive to oxidation. Stannous ion (Sn (II)) rapidly oxidizes to stannic ion (Sn (IV)) which is less bioactive. Therefore, maximizing the amount of tin in the stannous form (Sn (II)) is beneficial to providing these oral health benefits over the shelf life of the product.

Many efforts have been dedicated to suppress Sn(II) oxidation and achieve stable stannous formulations. One approach to suppressing Sn(II) oxidation is to use KNO₃ as a stabilizing agent (US 2013/0039867). It was found that at a low pH, SnF₂ solution becomes more stable and less prone to oxidation when KNO₃ salt is added to SnF₂. Although several methods of stabilizing stannous ion have been known in the art, both maximization and stabilization of tin in the stannous form (tin II) in oral care formulations has remained an ongoing challenge.

Accordingly, there exists a need for stabilized stannous formulations with improved oral health benefits.

### BRIEF SUMMARY

The present disclosure provides oral care compositions, e.g., toothpaste or mouthwash, comprising a stannous ion source (e.g., stannous fluoride), tetrasodium pyrophosphate and an antioxidant. In some embodiments, the antioxidant is selected from ascorbyl phosphate, ascorbate, butylated hydroxytoluene (BHT), and butylated hydroxyanisole (BHA). In some embodiments, the antioxidant is selected from ascorbyl phosphate and ascorbate. In some embodiments, the antioxidant is ascorbyl phosphate, e.g., sodium ascorbyl phosphate (SAP). In some embodiments, pH of the composition is from 6.5 to 7.5, e.g., from 6.6 to 7.4, from 6.7 to 7.3, from 6.8 to 7.2, from 6.9 to 7.1, or about 7.0. For example, the oral care composition may preferably be formulated to have a pH of about 5.5 to about 9, about 5.5 to about 8.5, about 5.5 to about 8, about 5.5 to about 7.5, about 5.5 to about 7, about 5.5 to about 6.5, about 5.5 to about 6; from about 6 to about 9, about 6 to about 8.5, about 6 to about 8, about 6 to about 7.5, about 6 to about 7, about 6 to about 6.5; from about 6.5 to about 9, about 6.5 to about 8.5, about 6.5 to about 8, about 6.5 to about 7.5, about 6.5 to about 7; from about 7 to about 9, about 7 to about 8.5, about 7 to about 8, about 7 to about 7.5; from about 7.5 to about 9, about 7.5 to about 8.5, about 7.5 to about 8; from about 8 to about 9, about 8 to about 8.5, or any range or subrange thereof.

The oral care composition may be formulated to have a weight ratio of the total amount of antioxidant to the total amount of stannous ion source and TSPP (antioxidant:(stannous ion source and TSPP)) is from about 1:1.5 to about 1:8. For example, the oral care composition may have a weight ratio of the total amount of antioxidant to the total amount of stannous ion source and TSPP of about 1:1.5 to about 1:8, about 1:1.5 to about 1:7, about 1:1.5 to about 1:6, about 1:1.5 to about 1:5.5, about 1:1.5 to about 1:5, about 1:1.5 to about 1:4.5; from about 1:2 to about 1:8, about 1:2 to about 1:7, about 1:2 to about 1:6, about 1:2 to about 1:5.5, about 1:2 to about 1:5, about 1:2 to about 1:4.5; from about 1:2.5 to about 1:8, about 1:2.5 to about 1:7, about 1:2.5 to about 1:6, about 1:2.5 to about 1:5.5, about 1:2.5 to about 1:5, about 1:2.5 to about 1:4.5; from about 1:3 to about 1:8, about 1:3 to about 1:7, about 1:3 to about 1:6, about 1:3 to about 1:5.5, about 1:3 to about 1:5, about 1:3 to about 1:4.5, or any range or subrange thereof. In some embodiments, the molar ratio of stannous:TSPP:antioxidant is 1:0.5-1.5:0.05-1, e.g., 1:0.7-1.3:0.05-1, 1:0.8-1.2:0.05-1, 1:0.9-1.1:0.05-1, 1:0.5-1.5:0.05-0.3, 1:0.7-1.3:0.05-0.3, 1:0.8-1.2:0.05-0.3, 1:0.9-1.1:0.05-0.3, 1:0.5-1.5:0.05-0.2, 1:0.7-1.3:0.05-0.2, 1:0.8-1.2:0.05-0.2, 1:0.9-1.1:0.05-0.2, 1:0.5-1.5:0.05-0.15, 1:0.7-1.3:0.05-0.15, 1:0.8-1.2:0.05-0.15, 1:0.9-1.1:0.05-0.15, 1:0.5-1.5:0.05-0.1, 1:0.7-1.3:0.05-0.1, 1:0.8-1.2:0.05-0.1, 1:0.9-1.1:0.05-0.1, 1:1:0.05-1, 1:1:0.05-0.3, 1:1:0.05-0.2, 1:1:0.05-0.15, 1:1:0.05-0.1, 1:1:0.3, 1:1:0.2, 1:1:0.15, 1:1:0.1 or 1:1:0.05.

The present disclosure further provide a methods comprising applying an effective amount of an oral care composition as disclosed herein to the oral cavity, e.g., by brushing, to a subject in need thereof, to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

The present disclosure further encompasses the use of an antioxidant in an oral care composition comprising a stannous ion source and tetrasodium pyrophosphate for increasing the stability of stannous ion in the composition. In some embodiments, the antioxidant is selected from ascorbyl phosphate, ascorbate, butylated hydroxytoluene (BHT), and butylated hydroxyanisole (BHA). In some embodiments, the antioxidant is selected from ascorbyl phosphate and ascorbate. In some embodiments, the antioxidant is ascorbyl phosphate, e.g., sodium ascorbyl phosphate. In some embodiments, pH of the composition is from 6.5 to 7.5, e.g., from 6.6 to 7.4, from 6.7 to 7.3, from 6.8 to 7.2, from 6.9 to 7.1, or about 7.0.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

FIG. 1 shows the infrared absorption spectra (850-12400 cm⁻¹ range) of SnF₂−TSPP solution with and without antioxidant addition as a function of aging at 60° C. Water spectrum was subtracted from each individual curve. The spectra are offset for clarity. The vertical dash lines guide the eye.

FIG. 2 displays the amount of Sn(II) remaining in the samples of SnF₂+TSPP with/without antioxidant (BHT, BHA, or SAP) upon aging at 60° C. according to the iodine titration data.

FIG. 3 shows ¹¹⁹Sn NMR spectra (left) and ³¹P NMR spectra (right) of the SnF₂-TSPP-SAP solution upon aging at 60° C.

FIG. 4 shows ¹¹⁹Sn NMR spectra (left) and ³¹P NMR spectra (right) of the SnF₂-TSPP-BHA solution upon aging at 60° C.

FIG. 5 shows ¹¹⁹Sn NMR spectra (left) and ³¹P NMR spectra (right) of the SnF₂-TSPP-BHT solution upon aging at 60° C.

FIG. 6 displays the relative amount of Sn(II) remaining in the samples of SnF₂+TSPP with/without antioxidant (BHT, BHA, or SAP) upon aging at 60° C. according to the ¹¹⁹Sn NMR data.

FIG. 7 shows the FTIR spectra of the SnF₂-TSPP solution in presence of SAP. The molar ratio of SAP/Sn was varied from 0 to 0.3, as indicated. Each sample is shown in fresh, 1 week and 2 weeks 60° C. aged state. The water spectrum was subtracted from each curve. The spectra are offset for clarity and the vertical dash lines guide the eye.

FIG. 8 displays the FTIR spectra of the SnF₂-TSPP solution in presence of sodium ascorbate. The molar ratio of ascorbate/Sn was varied from 0 to 0.3, as indicated. Each sample is shown in fresh, 1 week and 2 weeks 60° C. aged state. The water spectrum was subtracted from each curve. The spectra are offset for clarity and the vertical dash lines guide the eye.

FIG. 9 displays ¹¹⁹Sn NMR spectra of SnF₂-TSPP solution at pH 7.0 at various concentrations of SAP upon aging at 60° C.

FIG. 10 shows ³¹P NMR spectra of SnF₂-TSPP solution at pH 7.0 at various concentrations of SAP upon aging at 60° C.

FIG. 11A shows the relative amount of Sn(II) remaining in the samples (SnF₂-TSPP at pH 7.0 at various concentrations of sodium ascorbate) upon aging at 60° C. according to the ¹¹⁹Sn NMR data. FIG. 11B shows the amount of Sn(II) remaining in the samples (SnF₂-TSPP at pH 7.0 at various concentrations of sodium ascorbate) upon aging at 60° C. according to the iodine titration data.

FIG. 12 displays ¹¹⁹Sn NMR spectra of SnF₂-TSPP solution at pH 7.0 at various concentrations of sodium ascorbate upon aging at 60° C.

FIG. 13 shows ³¹P NMR spectra of SnF₂-TSPP solution at pH 7.0 at various concentrations of sodium ascorbate upon aging at 60° C.

FIG. 14A shows the relative amount of Sn(II) remaining in the samples (SnF₂-TSPP at pH 7.0 at various concentrations of sodium ascorbate) upon aging at 60° C. according to the ¹¹⁹Sn NMR data. FIG. 14B shows the amount of Sn(II) remaining in the samples (SnF₂-TSPP at pH 7.0 at various concentrations of sodium ascorbate) upon aging at 60° C. according to the iodine titration data.

FIG. 15 displays the head-space O₂ consumption monitored indirectly through differential pressure changes above the solutions containing stannous fluoride and tetrasodium pyrophosphate without and with SAP at 0.05 and 0.3 SAP/Sn molar ratios.

FIG. 16A displays ¹¹⁹Sn of SnF₂-TSPP solution at pH 7.0 at various concentrations of SAP.

FIG. 16B displays ³¹P NMR spectra of SnF₂-TSPP solution at pH 7.0 at various concentrations of SAP. FIG. 16C displays the ¹H NMR spectra of SAP at various concentrations in SnF₂-TSPP solution.

FIG. 17 shows the amount of Sn(II) remaining in the toothpastes of various concentrations (0%-1%) of SAP upon aging at 60° C.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Stannous ion (Sn (II)) rapidly oxidizes to stannic ion (Sn (IV)) which is less bioactive. In the present invention, it has been found that a high proportion of tin is present in the stannous form (Sn (II)) with aging when an antioxidant is added to a composition having a neural pH which contains a stannous ion source and tetrasodium pyrophosphate (TSPP). Without intending to be bound to any theory, it is believed that stannous ions are chelated by TSPP and antioxidants suppress the oxidation of stannous ion and stabilize the Sn(II)-pyrophosphate complex at neutral pH.

Among the tested antioxidants, sodium ascorbic phosphate (SAP) is a preferable antioxidant in stabilizing Sn(II). SAP is a stable, water-soluble form of vitamin C made from combining ascorbic acid with a phosphate and a salt. Vitamin C or ascorbic acid is a well-known antioxidant found naturally in many fruits and added to many supplements and cosmetic products. Consumers are aware of many benefits of vitamin C including its role in immune defense, skin repair, and free radical scavenging. However, Vitamin C is known to oxidize and become yellow, which reduces its benefits.

It has been surprisingly found that a low concentration of sodium ascorbyl phosphate or sodium ascorbate, e.g., corresponding to as little as 0.05-0.1, for antioxidant/Sn ratio can enhance stannous stability in aqueous solutions at and/or near neutral pH. The use of the minimal concentration of antioxidants (sodium ascorbyl phosphate or sodium ascorbate) is beneficial for the cost reduction and improved color stability of the products containing the antioxidants. This finding offers the use of the minimal concentration of antioxidants (ascorbyl phosphate or ascorbate) to improve the stability of Sn(II) in oral care products.

As used herein, “Vitamin C” may be ascorbic acid or derivatives thereof. Ascorbic acid exists as two enantiomers commonly denoted “1” (for “levo”) and “d” (for “dextro”). The “1” isomer is the one most often encountered. Ascorbic acid is also referred to as L(+)-ascorbic acid or l-ascorbic acid. The ascorbic acid derivatives may be or include, but are not limited to calcium ascorbate, calcium l-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-1-(+)-ascorbic acid dimer, sodium ascorbyl phosphate (also referred to as: ascorbic acid phosphate sodium salt, sodium l-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, or the like, or combinations thereof.

The present invention provides, in an aspect, an oral care composition (Composition 1.0), for example toothpaste or mouthwash, that comprises a stannous ion source (e.g., stannous fluoride), tetrasodium pyrophosphate and an antioxidant. For example, the invention includes:

1.1. Composition 1.0, wherein the composition comprises an antioxidant selected from ascorbyl phosphate, ascorbate (or ascorbic acid), butylated hydroxytoluene (BHT), and butylated hydroxyanisole (BHA), optionally wherein the antioxidant is selected from ascorbyl phosphate and ascorbate (or ascorbic acid), further optionally wherein the ascorbyl phosphate is sodium ascorbyl phosphate and the ascorbate is sodium ascorbate. The antioxidant may comprise one or more Vitamin C, such as those described herein. In some embodiments, the antioxidants consist of one or more Vitamin C, such as those disclosed herein. The antioxidant(s) may be present in the oral care composition in an amount from about 0.05 to about 5 wt. %, based on the total weight of the oral care composition. In some embodiments, the total amount of antioxidant(s) is from about 0.05 to about 0.1 wt. %, based on the total weight of the oral care composition.

1.2. Composition 1.0 or 1.1, wherein the antioxidant is ascorbyl phosphate, optionally wherein the antioxidant is sodium ascorbyl phosphate.

1.3. Any of the preceding compositions, wherein the molar ratio of stannous ion source:TSPP:antioxidant is 1:0.5-1.5:0.05-1, optionally wherein the molar ratio of stannous ion source:TSPP:antioxidant is 1:0.7-1.3:0.05-1, 1:0.8-1.2:0.05-1, 1:0.9-1.1:0.05-1, 1:0.5-1.5:0.05-0.3, 1:0.7-1.3:0.05-0.3, 1:0.8-1.2:0.05-0.3, 1:0.9-1.1:0.05-0.3, 1:0.5-1.5:0.05-0.2, 1:0.7-1.3:0.05-0.2, 1:0.8-1.2:0.05-0.2, 1:0.9-1.1:0.05-0.2, 1:0.5-1.5:0.05-0.15, 1:0.7-1.3:0.05-0.15, 1:0.8-1.2:0.05-0.15, 1:0.9-1.1:0.05-0.15, 1:0.5-1.5:0.05-0.1, 1:0.7-1.3:0.05-0.1, 1:0.8-1.2:0.05-0.1, 1:0.9-1.1:0.05-0.1, 1:1:0.05-1, 1:1:0.05-0.3, 1:1:0.05-0.2, 1:1:0.05-0.15, 1:1:0.05-0.1, 1:1:0.3, 1:1:0.2, 1:1:0.15, 1:1:0.1 or 1:1:0.05.

1.4. Any of the preceding compositions, wherein the composition comprises potassium nitrate.

1.5. Any of Compositions 1.0 to 1.3, wherein the composition does not contain potassium nitrate.

1.6. Any of the preceding compositions, wherein the stannous ion source is selected from the group consisting of stannous fluoride, stannous gluconate, stannous phosphate, stannous pyrophosphate, stannous acetate, stannous sulfate, stannous chloride and a combination thereof.

1.7. Any of the preceding compositions, wherein the stannous ion source is stannous fluoride.

1.8. Composition 1.7, wherein the composition further comprises a stannous ion source which is not stannous fluoride.

1.9. Any of the preceding compositions, wherein the stannous ion source is present in an amount of from 0.01% to 10%, e.g., from 0.1% to 5%, from 1% to 5%, from 1.5 to 4%, from 0.1% to 1%, from 0.1% to 0.2%, from 0.2% to 0.8%, from 0.2% to 0.5%, from 0.3% to 0.6%, or from 0.4% to 0.5% by weight of the composition.

1.10. Any of the preceding compositions, wherein the composition further comprises a zinc source. The zinc source may be a zinc ion source.

1.11. Composition 1.10, wherein the zinc source (e.g., a zine ion source) is selected from the group consisting of zinc oxide, zine sulfate, zinc chloride, zinc citrate, zinc lactate, zinc gluconate, zinc malate, zinc tartrate, zinc carbonate, zinc phosphate, and a combination thereof.

1.12. Combination 1.10 or 1.11, wherein the zinc source is present an amount of from 0.01% to 5%, e.g., 0.1% to 4%, or 0.5% to 3%, by weight of the composition. For example, the amount of zinc source present in the oral care composition may be from about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %, about 3 to about 5 wt. %, or any range or subrange thereof, based on the total weight of the oral care composition.

1.13. Any of Compositions 1.10 to 1.12, wherein the composition comprises zinc oxide.

1.14. Composition 1.13, wherein zinc oxide is present in an amount of from 0.5% to 2%, e.g., from 0.5% to 1.5%, from 0.8% to 1.3%, from 1% to 1.2%, from 1.1% to 1.3%, about 1%, or about 1.2% by weight based on the total weight of the oral care composition.

1.15. Any of Compositions 1.10 to 1.12, wherein the oral care composition comprises zinc oxide and zinc citrate.

1.16. Composition 1.15, wherein zinc oxide is present in an amount of from 0.5% to 2%, e.g., from 0.5% to 1.5%, from 0.8% to 1.3%, from 1% to 1.2%, from 1.1% to 1.3%, about 1%, or about 1.2% by weight based on the total weight of the oral care composition and zinc citrate is present in an amount of from 0.1% to 1%, from 0.25% to 0.75%, from 0.3% to 0.6%, about 0.5% by weight based on the total weight of the oral care composition.

1.17. Composition 1.16, wherein zinc oxide is present in an amount of about 1% by weight based on the total weight of the oral care composition and zinc citrate is present in an amount of about 0.5% by weight based on the total weight of the oral care composition.

1.18. Any of the preceding compositions, wherein the composition comprises zinc phosphate.

1.19. Composition 1.18, wherein zinc phosphate is present in an amount of from 0.5% to 2%, e.g., from 0.5% to 1.5%, from 0.8% to 1.3%, from 1% to 1.2%, from 1.1% to 1.3%, about 1%, or about 1.2% by weight based on the total weight of the oral care composition.

1.20. Any of the preceding compositions, wherein the composition comprises a fluoride ion source is selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and a combination thereof.

1.21. Composition 1.20, wherein the fluoride ion source is stannous fluoride.

1.22. Composition 1.21, wherein the oral care composition further comprises a fluoride ion source which is not stannous fluoride.

1.23. The composition of Composition 1.20 to 1.22, wherein the oral care composition comprises fluoride ion sources in amounts sufficient to supply 25 ppm to 5,000 ppm of fluoride ions, generally at least 500 ppm, e.g., 500 to 2000 ppm, e.g., 1000 ppm to 1600 ppm, e.g., 1450 ppm.

1.24. Any of the preceding Compositions, wherein the oral care composition comprises a basic amino acid in free or salt form.

1.25. Composition 1.24, wherein the basic amino acid comprises one or more of arginine, lysine, citrulline, ornithine, creatine, histidine, diaminobutyric acid, diaminopropionic acid, salts thereof, or combinations thereof.

1.26. Composition 1.24 or 1.25, wherein the basic amino acid has the L-configuration.

1.27. Any of Compositions 1.24 to 1.26, wherein the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight based on the total weight of the oral care composition, being calculated as free base form.

1.28. Any of Compositions 1.24-1.27, wherein the basic amino acid comprises arginine.

1.29. Composition 1.28, wherein the basic amino acid comprises arginine bicarbonate, arginine phosphate, arginine sulfate, arginine hydrochloride or combinations thereof, optionally wherein the basic amino acid is arginine bicarbonate. In some embodiments, the basic amino acid comprise arginine (e.g., in free form). In at least one embodiment, the basic amino acid comprises L-arginine.

1.30. Any of the preceding compositions, wherein the oral care composition comprises one or more thickeners, for example thickening silicas.

1.31. Any of the preceding compositions, wherein the oral care composition comprises a foaming agent, for example a betaine, for example cocamidopropyl betaine.

1.32. Any of the preceding compositions, wherein the oral care composition comprises a chelator, optionally wherein the chelator is selected from citrate and EDTA.

1.33. Any of the preceding compositions, wherein the oral care composition comprises ingredients selected from one or more of buffering agents, humectants, surfactants, gum strips or fragments, breath fresheners, flavoring, fragrance, coloring, antibacterial agents, whitening agents, agents that interfere with or prevents bacterial attachment, calcium sources, and potassium salts.

1.34. Any of the preceding compositions, wherein pH of the oral care composition is from 6.5 to 7.5, optionally wherein pH of the oral care composition is from 6.6 to 7.4, e.g., from 6.7 to 7.3, from 6.8 to 7.2, from 6.9 to 7.1, or about 7.0. For example, the oral care composition may preferably be formulated to have a pH of about 5.5 to about 9, about 5.5 to about 8.5, about 5.5 to about 8, about 5.5 to about 7.5, about 5.5 to about 7, about 5.5 to about 6.5, about 5.5 to about 6; from about 6 to about 9, about 6 to about 8.5, about 6 to about 8, about 6 to about 7.5, about 6 to about 7, about 6 to about 6.5; from about 6.5 to about 9, about 6.5 to about 8.5, about 6.5 to about 8, about 6.5 to about 7.5, about 6.5 to about 7; from about 7 to about 9, about 7 to about 8.5, about 7 to about 8, about 7 to about 7.5; from about 7.5 to about 9, about 7.5 to about 8.5, about 7.5 to about 8; from about 8 to about 9, about 8 to about 8.5, or any range or subrange thereof.

1.35. Any of the preceding compositions, wherein the oral care composition is a dentifrice, a toothpaste, a gel, a mouthwash, a mouth rinse, a powder, a cream, a strip, a gum, bead, film, or floss.

1.36. Composition 1.35, wherein the oral care composition is a toothpaste.

1.37. Composition 1.35, wherein the oral care composition is a gel.

1.38. Composition 1.35, wherein the oral care composition is a mouthwash.

1.39. Any of the preceding compositions for use to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

The oral care composition may comprise a stannous ion source, tetrasodium pyrophosphate, and an antioxidant agent. Stannous ion sources are well known in the art and may be incorporated into the compositions of the present invention. In some embodiments, the stannous ion source is selected from the group consisting of stannous fluoride, stannous gluconate, stannous phosphate, stannous pyrophosphate, stannous acetate, stannous sulfate, stannous chloride and a combination thereof. In some embodiments, the stannous ion source is present in an amount of from 0.01% to 10%, e.g., from 0.1% to 5%, from 1% to 5%, from 1.5 to 4%, from 0.1% to 1%, from 0.1% to 0.2%, from 0.2% to 0.8%, from 0.2% to 0.5%, from 0.3% to 0.6%, or from 0.4% to 0.5%, by weight, based on the total weight of the oral care composition. In certain embodiments, the stannous ion source is stannous fluoride. In some embodiments, stannous fluoride is present in an amount of from 0.01% to 10%, e.g., from 0.5% to 7%, from 1% to 5%, from 1.5 to 4%, from 0.1% to 1%, from 0.1% to 0.2 from 0.2% to 0.8%, from 0.2% to 0.5%, from 0.3% to 0.6%, or from 0.4% to 0.5% by weight based on the total weight of the oral care composition. In some embodiments, the oral care composition may contain a stannous ion source which is not stannous fluoride.

The oral care composition typically comprises an antioxidant agent. Antioxidants are compounds that have the ability to scavenge free radicals and thus slow down or suppress the oxidation processes by disrupting the radical chain reactions and inhibiting the formation of strong oxidation species. The one or more antioxidant(s) may comprise a Vitamin C, such as any of the Vitamin Cs disclosed above. The antioxidant may comprise one or more Vitamin C, such as those described above. In some embodiments, the antioxidants consist of one or more Vitamin C, such as those disclosed herein. For example, the antioxidants may consists of one or more of butylated hydroxytoluene (BHT), butylated hydroxyanisole (BHA), ascorbic acid, calcium ascorbate, calcium l-ascorbate dihydrate, magnesium ascorbate, potassium ascorbate, magnesium L-ascorbyl phosphate, L-ascorbic acid 2-phosphate sesquimagnesium salt hydrate, (+) sodium L-ascorbate, dehydro-1-(+)-ascorbic acid dimer, sodium ascorbyl phosphate, ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, and a combination of two or more thereof. The Vitamin C may be an ascorbate. The ascorbate may be selected from calcium ascorbate, calcium l-ascorbate dihydrate, magnesium ascorbate, potassium ascorbate, magnesium L-ascorbyl phosphate (also referred to as: magnesium ascorbate phosphate or ascorbic acid phosphate magnesium salt), and a combination of two or more thereof.

In some embodiments, the antioxidant is selected from ascorbyl phosphate, ascorbate (or ascorbic acid), butylated hydroxytoluene (BHT), and butylated hydroxyanisole (BHA). In some embodiments, the antioxidant is selected from an ascorbyl phosphate, an ascorbate, and acids thereof (such as, ascorbic acid). The antioxidants may be in free or salt form. In some embodiment, ascorbyl phosphate is sodium ascorbyl phosphate and ascorbate is sodium ascorbate. In certain embodiments, the antioxidant is ascorbyl phosphate, e.g., sodium ascorbyl phosphate.

The antioxidant(s) may be present in the oral care composition in an amount from about 0.05 to about 5 wt. %, based on the total weight of the oral care composition. For example, the total amount of antioxidant(s) in the oral care composition may be from about 0.05 to about 5 wt. %, about 0.05 to about 4 wt. %, about 0.05 to about 3 wt. %, about 0.05 to about 2 wt. %, about 0.05 to about 1.5 wt. %, about 0.05 to about 1.2 wt. %, about 0.05 to about 1 wt. %, about 0.05 to about 0.8 wt. %, about 0.05 to about 0.6 wt. %; from about 0.1 to about 5 wt. %, about 0.1 to about 4 wt. %, about 0.1 to about 3 wt. %, about 0.1 to about 2 wt. %, about 0.1 to about 1.5 wt. %, about 0.1 to about 1.2 wt. %, about 0.1 to about 1 wt. %, about 0.1 to about 0.8 wt. %, about 0.1 to about 0.6 wt. %; from about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1.2 wt. %, about 0.5 to about 1 wt. %, about 0.5 to about 0.8 wt. %; from about 0.8 to about 5 wt. %, about 0.8 to about 4 wt. %, about 0.8 to about 3 wt. %, about 0.8 to about 2 wt. %, about 0.8 to about 1.5 wt. %, about 0.8 to about 1.2 wt. %, about 0.8 to about 1 wt. %; from about 1 to about 5 wt. %, about 1 to about 4 wt. %, about 1 to about 3 wt. %, about 1 to about 2 wt. %, about 1 to about 1.5 wt. %, about 1 to about 1.2 wt. %; from about 1.2 to about 5 wt. %, about 1.2 to about 4 wt. %, about 1.2 to about 3 wt. %, about 1.2 to about 2 wt. %, about 1.2 to about 1.5 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the oral care composition. In some embodiments, the total amount of antioxidant(s) is from about 0.05 to about 0.1 wt. %, based on the total weight of the oral care composition.

The antioxidant is present in an effective amount to stabilize stannous ion. In some embodiments, the molar ratio of stannous:TSPP:antioxidant is 1:0.5-1.5:0.05-1, e.g., 1:0.7-1.3:0.05-1, 1:0.8-1.2:0.05-1, 1:0.9-1.1:0.05-1, 1:0.5-1.5:0.05-0.3, 1:0.7-1.3:0.05-0.3, 1:0.8-1.2:0.05-0.3, 1:0.9-1.1:0.05-0.3, 1:0.5-1.5:0.05-0.2, 1:0.7-1.3:0.05-0.2, 1:0.8-1.2:0.05-0.2, 1:0.9-1.1:0.05-0.2, 1:0.5-1.5:0.05-0.15, 1:0.7-1.3:0.05-0.15, 1:0.8-1.2:0.05-0.15, 1:0.9-1.1:0.05-0.15, 1:0.5-1.5:0.05-0.1, 1:0.7-1.3:0.05-0.1, 1:0.8-1.2:0.05-0.1, 1:0.9-1.1:0.05-0.1, 1:1:0.05-1, 1:1:0.05-0.3, 1:1:0.05-0.2, 1:1:0.05-0.15, 1:1:0.05-0.1, 1:1:0.3, 1:1:0.2, 1:1:0.15, 1:1:0.1 or 1:1:0.05.

In some embodiments, the composition of the present invention comprises potassium nitrate. In other embodiments, the composition does not contain potassium nitrate.

The oral care compositions typically comprises one or more nitrate ion source (e.g., potassium nitrate). The nitrate ion source(s) (e.g., potassium nitrate) may be present in the oral care composition in an amount from about 0.1 to about 5 wt. %, based on the total weight of the oral care composition. In some instances, the amount of nitrate ion present in the oral care composition may be from about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The oral care compositions may be formulated to have a molar ratio of nitrate ions to stannous ions, both measured as free ions, that is about 2:1 or less. For example, the oral care composition may have a molar ratio of nitrate ions to stannous ions, both measured as free ions, of from about 0.5:1 to about 2:1, about 0.5:1 to about 1.8:1, about 0.5:1 to about 1.6:1, about 0.5:1 to about 1.4:1, about 0.5:1 to about 1.2:1, about 0.5:1 to about 1:1; from about 0.7:1 to about 2:1, about 0.7:1 to about 1.8:1, about 0.7:1 to about 1.6:1, about 0.7:1 to about 1.4:1, about 0.7:1 to about 1.2:1, about 0.7:1 to about 1:1; from about 0.9:1 to about 2:1, about 0.9:1 to about 1.8:1, about 0.9:1 to about 1.6:1, about 0.9:1 to about 1.4:1, about 0.9:1 to about 1.2:1, about 0.9:1 to about 1:1, or any range or subrange thereof. In some embodiments, the oral care composition is formulated to have a molar ratio of nitrate ions to stannous ions, both measured as free ions, of about 1:1.

The tetrasodium pyrophosphate may be present in the oral care composition in an amount from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The oral care composition of the present invention can be in the form of any oral care formulations, including dentifrice, toothpaste, gel, mouthwash, mouth rinse, powder, cream, strip, gum, bead, film, floss or any other known in the art. In some embodiments, the oral care composition is a toothpaste or gel. In other embodiments, the oral care composition is a mouthwash or mouth rinse.

The oral care composition of the present invention may be a single phase oral care composition. For example, all the components of the oral care composition may be maintained together with one another in a single phase and/or vessel. For example, all the components of the oral care composition may be maintained in a single phase, such as a single homogenous phase. In another embodiment, the oral care composition may be a multi-phase oral care composition.

The oral care composition of the present invention may contain an orally acceptable carrier. As used herein, an “orally acceptable carrier” refers to a material or combination of materials that are safe for use in the compositions of the invention, commensurate with a reasonable benefit/risk ratio. Such materials include but are not limited to, for example, water, humectants, ionic active ingredients, buffering agents, anticalculus agents, abrasive polishing materials, peroxide sources, alkali metal bicarbonate salts, surfactants, titanium dioxide, coloring agents, flavor systems, sweetening agents, antimicrobial agents, herbal agents, desensitizing agents, stain reducing agents, and mixtures thereof. Such materials are well known in the art and are readily chosen by one skilled in the art based on the physical and aesthetic properties desired for the compositions being prepared. In some embodiment, the orally acceptable carrier may include an orally acceptable solvent. Illustrative solvents may include, but are not limited to, one or more of ethanol, phenoxyethanol, isopropanol, water, cyclohexane, methyl glycol acetate, benzyl alcohol, or the like, or any mixture or combination thereof. In a particular embodiment, the orally acceptable solvent includes benzyl alcohol.

Water may be present in the oral care composition of the present invention. Water employed in the preparation of commercial oral compositions should be deionized and free of organic impurities. Water commonly makes up the balance of the compositions and includes about 10% to about 90%, about 10% to about 80%, about 20% to about 60%, about 20% to 40%, about 10% to about 30%, about 20% to 30%, about 25% to 35%, about 70% to 90%, or about 80% to 90%, by weight based on the total weight of the oral compositions. This amount of water includes the free water which is added plus that amount which is introduced with other materials such as with sorbitol or any components of the invention.

The composition of the present invention may comprise a pH adjuster. For example, the compositions may comprise an acid or base in an amount sufficient to adjust the pH of the compositions. In some embodiments, pH of the composition is neutral. The desired pH of the composition may be from 6.5 to 7.5, e.g., from 6.6 to 7.4, from 6.7 to 7.3, from 6.8 to 7.2, from 6.9 to 7.1, or about 7.0.

In some embodiments, the composition of the present invention comprises a fluoride ion source. Preferably, the fluoride ion source is stannous fluoride. In some embodiments, the composition may contain other fluoride which is not stannous fluoride. Representative fluoride ion sources include, but are not limited to, sodium fluoride, potassium fluoride, sodium monofluorophosphate, sodium fluorosilicate, ammonium fluorosilicate, amine fluoride, ammonium fluoride, and combinations thereof. In some embodiments, the composition may contain fluoride ion sources in amounts sufficient to supply 25 ppm to 5,000 ppm of fluoride ions, generally at least 500 ppm, e.g., 500 to 2000 ppm, e.g., 1000 ppm to 1600 ppm, e.g., 1450 ppm. Fluoride ion sources may be added to the compositions of the invention at a level of 0.01 to 10%, e.g., 0.03% to 5%, or 0.1% to 1%, by weight based on the total weight of the oral care composition. However, it is to be understood that the weights of fluoride salts to provide the appropriate level of fluoride ion will obviously vary based on the weight of the counter ion in the salt, and one of skill in the art may readily determine such amounts.

The composition of the present invention may include one or more zinc ion sources. Zinc ions have been found to help in the reduction of gingivitis, plaque, sensitivity, and improved breath benefits. The zinc ion source can be a soluble or sparingly soluble compound of zinc with inorganic or organic counter ions. Examples include zinc oxide, zinc sulfate, zinc chloride, zinc citrate, zinc lactate, zinc gluconate, zinc malate, zinc tartrate, zinc carbonate, and zine phosphate. In some embodiments, the zinc ion source is present in an amount of from 0.01% to 5%, e.g., 0.1% to 4%, or 1% to 3%, by weight of the oral care composition.

In preferred embodiments, the composition comprises zinc oxide. Zinc oxide may be present in an amount of 0.5% to 2%, e.g., 0.5% to 1.5%, about 1% or about 1.2% by weight of the oral care composition. In some embodiments, the composition comprises zinc oxide and zinc citrate. The compositions may comprise zinc oxide in an amount of 0.5% to 2%, e.g., 0.5% to 1.5%, about 1% or about 1.2% by weight of the composition and zinc citrate in an amount of 0.1%-1%, 0.25-0.75%, about 0.5% by weight of the composition. In some embodiments, the compositions comprise zinc oxide in an amount of 1% by weight of the composition and zinc citrate in an amount of 0.5% by weight based on the total weight of the composition.

In some embodiments, the composition comprises zinc phosphate. In some embodiments, the composition may comprise zinc phosphate in an amount of 0.5% to 2%, e.g., 0.5% to 1.5%, about 1% or about 1.2% by weight based on the total weight of the composition.

The oral care composition of the present invention may comprise a basic amino acid in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrulline, ornithine, creatine, histidine, diaminobutyric acid, diaminopropionic acid, salts thereof or combinations thereof. In a particular embodiment, the basic amino acids are selected from arginine, lysine, citrulline, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the oral care composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40% solution of the basic amino acid, such as arginine bicarbonate or alternatively called as arginine carbamate. In some embodiments, the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight based on the total weight of the composition, being calculated as free base form.

The composition of the present invention may include other active ingredients. The active ingredients include, for example, anti-bacterial active agents, anti-tartar agents, anti-caries agents, anti-inflammatory agents, anti-sensitivity agents, enzymes, nutrients, and the like. Actives useful herein are optionally present in the compositions of the present invention in safe and effective amounts that are sufficient to have the desired therapeutic or prophylactic effect in the human or lower animal subject to whom the active is administered, without undue adverse side effects (such as toxicity, irritation, or allergic response), commensurate with a reasonable risk/benefit ratio when used in the manner of this invention. The specific safe and effective amount of the active will vary with such factors as the particular condition being treated, the physical condition of the subject, the nature of concurrent therapy (if any), the specific active used, the specific dosage form, the carrier employed, and the desired dosage regimen.

In some embodiments, the oral care compositions may include one or more abrasives or an abrasive system including one or more abrasives. As used herein, the term “abrasive” may also refer to materials commonly referred to as “polishing agents”. Any orally acceptable abrasive may be used, but preferably, type, fineness (particle size), and amount of the abrasive may be selected such that the tooth enamel is not excessively abraded in normal use of the oral care composition. The one or more abrasives may have a particle size or D50 of less than or equal to about 10 μm, less than or equal to about 8 μm, less than or equal to about 5 μm, or less than or equal to about 3 μm. The one or more abrasives may have a particle size or D50 of greater than or equal to about 0.01 μm, greater than or equal to about 0.05 μm, greater than or equal to about 0.1 μm, greater than or equal to about 0.5 μm, or greater than or equal to about 1 μm. Illustrative abrasives may include, but are not limited to, metaphosphate compounds, phosphate salts (e.g., insoluble phosphate salts), such as sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate, calcium carbonate (e.g., precipitated calcium carbonate and/or natural calcium carbonate), magnesium carbonate, hydrated alumina, silica, zirconium silicate, aluminum silicate including calcined aluminum silicate, polymethyl methacrylate, or the like, or mixtures and combinations thereof. In some embodiments, the oral care composition comprises a silica abrasive. In some embodiments, the silica abrasive is present in an amount of from 10% to 30%, e.g., 10% to 20%, 15% to 25%, or about 15%, by weight, based on the total weight of the composition. In some embodiments, the oral care composition comprises a calcium-free silica abrasive. In some embodiments, the composition is substantially free of calcium, e.g., comprises less than 2%, less than 1%, less than 0.5%, or less than 0.1% calcium by weight of the oral care composition.

The oral care composition of the present invention may include one or more agents to increase the amount of foam that is produced when the oral cavity is brushed. Such foaming agents are known to those of skill in the art. Illustrative examples of agents that increase the amount of foam include, but are not limited to polyoxyethylene and certain polymers including, but not limited to, alginate polymers. The polyoxyethylene may increase the amount of foam and the thickness of the foam. Polyoxyethylene is also commonly known as polyethylene glycol (“PEG”) or polyethylene oxide. The polyoxyethylenes suitable for this invention will have a molecular weight of 200,000 to 7,000,000, e.g., 600,000 to 2,000,000 or 800,000 to 1,000,000. The polyoxyethylene may be present in an amount of 1% to 90%, e.g., 5% to 50% or 10% to 20%, by weight of the composition. The dosage of foaming agent in the composition (i.e., a single dose) is 0.01 to 0.9%, e.g., 0.05 to 0.5% or 0.1 to 0.2%, by weight based on the total weight of the oral care composition.

The oral care composition of may include at least one surfactant or solubilizer. Suitable surfactants include neutral surfactants (such as polyoxyethylene hydrogenated castor oil or fatty acids of sugars), anionic surfactants (such as sodium lauryl sulfate), cationic surfactants (such as the ammonium cation surfactants) or zwitterionic surfactants. These surfactants or solubilizers may be present in amounts of typically 0.01% to 2%; or from 1% to 2%; or about 1.5%, by weight based on the total weight of the oral care composition.

The oral care composition of the present invention may include chelators. In some embodiments, the composition comprises a chelator selected from citrate and EDTA.

The oral care composition of the present invention may include a sweetener such as, e.g., saccharin, for example sodium saccharin, acesulfame, neotame, cyclamate or sucralose; natural high-intensity sweeteners such as thaumatin, stevioside or glycyrrhizin; or such as sorbitol, xylitol, maltitol or mannitol. One or more of such sweeteners may be present in an amount of from 0.005% to 5% by weight, for example 0.01% to 1%, for example 0.01% to 0.5%, by weight based on the total weight of the oral care composition.

The oral care composition of the present invention may include one or more colorants. Colorants may include pigments, dyes, lakes and agents imparting a particular color or visual quality to the composition. Any orally acceptable colorant can be used. One or more colorants may optionally be present in the oral care compositions in an amount of from 0.001% to 2%, for example from 0.001% to 0.01%, for example from 0.001% to 0.005%, by weight based on the total weight of the oral care composition, of the oral composition.

The oral care composition of the present invention may include one or more humectants. Humectants can reduce evaporation and also contribute towards preservation by lowering water activity, and can also impart desirable sweetness or flavor to compositions. Suitable humectants include edible polyhydric alcohols such as glycerin, sorbitol, xylitol, propylene glycol as well as other polyols and mixtures of these humectants. Other useful materials may also include orally acceptable alcohols, or polymers, e.g., such as polyvinylmethyl ether maleic acid copolymers, polysaccharides (e.g. cellulose derivatives, for example carboxymethyl cellulose, or polysaccharide gums, for example xanthan gum or carrageenan gum). In some embodiments, the humectant can be present in an amount of from 20% to 60%, for example from 30% to 50%, for example from 40% to 45%, by weight of the oral care composition.

The oral care composition of the present invention may include a preservative. Suitable preservatives include, for example, sodium benzoate, potassium sorbate, methylisothiazolinone, paraben preservatives, for example methyl p-hydroxybenzoate, propyl p-hydroxybenzoate, and mixtures thereof.

The oral care composition may include a flavoring agent. Suitable flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent is typically incorporated in the oral composition at a concentration of 0.01 to 3% by weight based on the total weight of the oral care composition.

The oral care compositions can be manufactured following standard formulation procedure. For example, the toothpaste compositions can be manufactured as follows. Polymer gums are dispersed in glycerin with gentle stirring to make completely homogeneous gel phase. A premix is prepared by dissolving stannous fluoride and sodium saccharin in formula amounts of water. The premix solution is added to the gel phase and mixed for 12-15 minutes. Potassium nitrate, Chelator (citrate or EDTA), antioxidant (quercetin and catechol), tetrasodium pyrophosphate (TSPP). silica, zinc oxide and/or titanium dioxide are added to the mixture and mixed at low speed for 3-5 minutes for proper mixing. The mixture is then mixed at an increased speed under vacuum for 25-30 minutes to create a smooth dentifrice. Surfactants and flavoring agents are added to the oral care composition and mixed at full speed under vacuum for 12-15 minutes until homogeneous.

In another aspect, provide is a method to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, comprising applying an effective amount of any of dentifrice compositions as disclosed herein to the oral cavity of a subject in need thereof.

In another aspect, provided is a method to improve oral health comprising applying an effective amount of any of oral care compositions as disclosed herein to the oral cavity of a subject in need thereof.

In another aspect, provided is the use of any of oral care compositions as disclosed herein to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, in a subject in need thereof.

In another aspect, provided is the use of an antioxidant in an oral care composition comprising a stannous ion source and tetrasodium pyrophosphate for increasing the stability of stannous ion in the composition. In some embodiments, the antioxidant is selected from ascorbyl phosphate, ascorbate, butylated hydroxytoluene (BHT), and butylated hydroxyanisole (BHA). In some embodiments, the antioxidant is selected from ascorbyl phosphate and ascorbate. In some embodiments, the antioxidant is ascorbyl phosphate, e.g., sodium ascorbyl phosphate. In some embodiments, pH of the composition is from 6.5 to 7.5, e.g., from 6.6 to 7.4, from 6.7 to 7.3, from 6.8 to 7.2, from 6.9 to 7.1, or about 7.0.

The following examples are further illustrative of the preferred embodiments, but it is understood that the invention is not limited thereto.

### EXAMPLES

### Example 1

The effect of antioxidants on the stannous stability in the presence of tetrasodium pyrophosphate (TSPP) was examined. Samples of Sn(II) were prepared by dissolving TSPP and SnF₂ in water with the final concentration of SnF₂ equal to 2 wt. %. Antioxidant (butylated hydroxytoluene (BHT), butylated hydroxyanisole (BHA), or sodium ascorbyl phosphate (SAP)) was added to the samples with the molar ratio of SnF₂:TSPP:antioxidant equal to 1:1:0.3. The pH of the solutions was adjusted with NaOH to pH 7. The solutions were not fully clear due to the high concentration of SnF₂ and limited solubility of certain antioxidants. Some amount of the precipitate/undissolved material was evident in the vials. Table 1 lists the amounts of raw materials used in preparation of solutions.

TABLE 1
Raw materials and their quantities used in preparation of samples

| | SnF₂ | TSPP | Antioxidant | H₂O | |
|---|---|---|---|---|---|
| Sample | (g) | (g) | (g) | (g) | pH |
| SnF₂-TSPP-BHT | 2.0033 | 3.3940 | 0.8441 | 93.7370 | 7.01 |
| SnF₂-TSPP-BHA | 2.0101 | 3.3946 | 0.6921 | 94.9618 | 7.00 |
| SnF₂-TSPP-SAP | 2.0037 | 3.3940 | 1.2425 | 93.3831 | 7.01 |

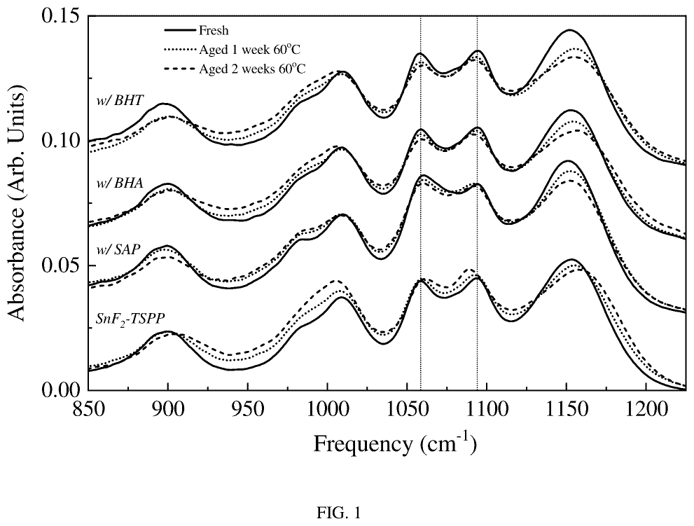

Fourier transform infrared spectroscopy (FTIR): Infrared spectra were collected using a Bruker Vertex 70 FTIR spectrometer (Bruker Optics, Billerica, MA) equipped with a GladiATR diamond ATR accessory (Pike technologies, Madison, WI). The spectral range was 80-4000 cm⁻¹ and a resolution of 4 cm⁻¹ was used. All measurements were carried out at room temperature. The absorption spectrum of solution containing SnF₂ and TSPP (herein after referred to as SnF₂-TSPP) in the 850-1240 cm⁻¹ region at different time points: fresh, 1 week and 2 weeks aged at 60° C. was examined. The result is shown in FIG. 1. Clear changes in the pyrophosphate bands of the stannous-pyrophosphate complex were observed with aging. The transformation of the complex was manifested in the peak shifts and intensity changes of phosphate bands. As an example, a doublet with peaks near 1058 and 1094 cm⁻¹ exhibited an inward band shifting towards a greater overlap of the two features after 2 weeks at 60° C., while the 900 and 1153 cm⁻¹ bands displayed a blue-shift with sample aging. The behavior of these absorption peaks was examined and compared with the addition of various antioxidants to the SnF₂-TSPP solution. When SAP, BHA, or BHT was added to stannous solution, phosphate vibrational bands displayed more modest changes with aging. Among the samples, the solution with SAP showed the smallest change after aging for 2 weeks at 60° C., suggesting better stability of stannous-pyrophosphate complex and slower Sn(II) oxidation in the presence of SAP. The effect of BHA and BHT on stannous complex was similar. BHA and BHT improved the stability of stannous-pyrophosphate complex but were less effective than SAP.

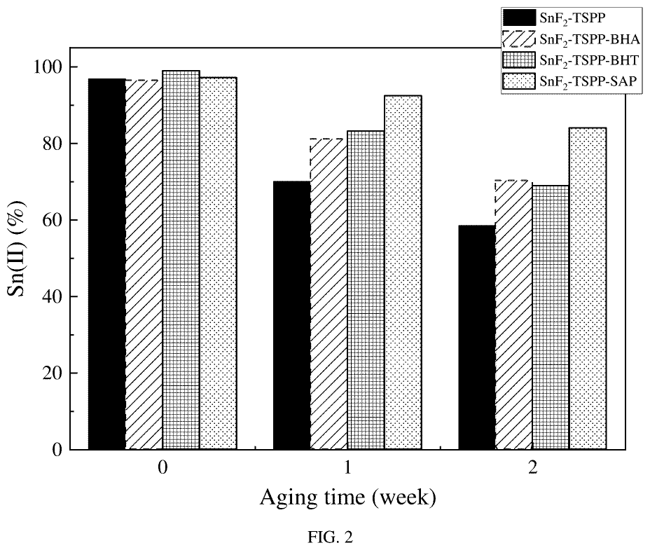

Iodine titration: The stability of Sn (II) was further examined by iodine titration assay. Titration measurements were performed through indirect titration. 2 M citric acid solution was added to the samples in excess and 0.1 N Iodine was added until the solutions turned light brown. The samples were covered with parafilm and aluminum foil and left mixing in the dark for 2 hours. Then, titration with 0.1 N sodium thiosulfate standard was performed using a 25 mL Titrette Bottletop Burette (BrandTech Scientific, Inc., Essex, CT, USA) until the solutions turned clear. Sn(II) concentration was calculated based on the amount of sodium thiosulfate and iodine used in the reaction. The amount of Sn(II) present in the SnF₂-TSPP solution at different time points: fresh, 1 week and 2 weeks aged at 60° C. with and without the antioxidant (SAP, BHA, or BHT) was measured. The result is shown in FIG. 2. The result is in good agreement with the FTIR result discussed above. The amount of Sn(II) was highest in the sample with SAP after 2 weeks of 60° C. aging. BHT and BHA showed similar Sn(II) recovery and displayed a positive effect on Sn(II) stability compared to SnF₂-TSPP solution alone.

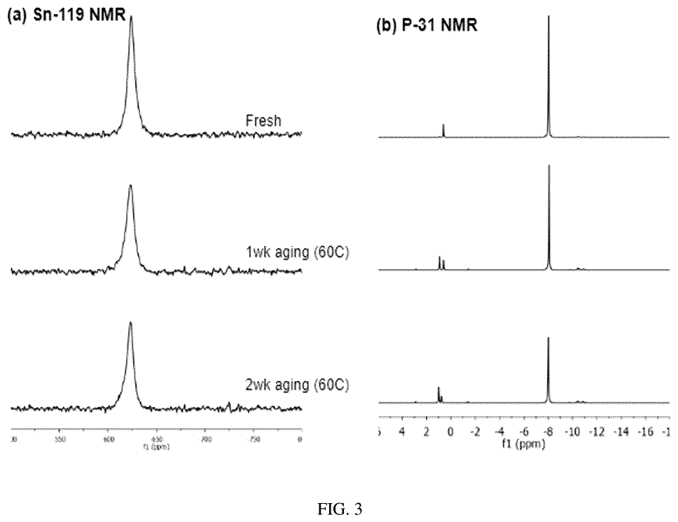

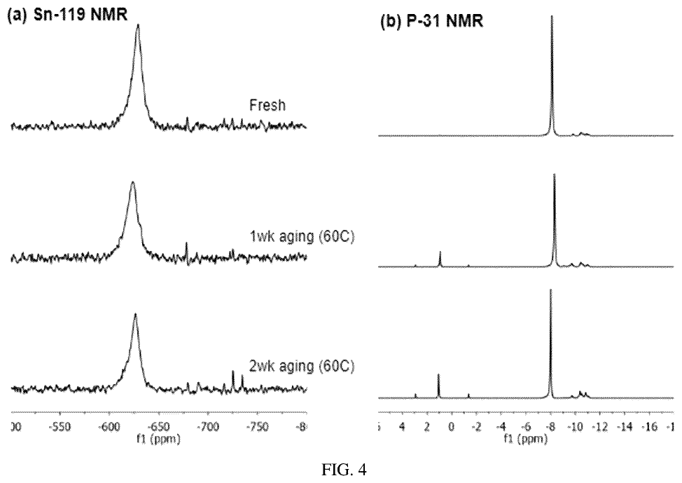

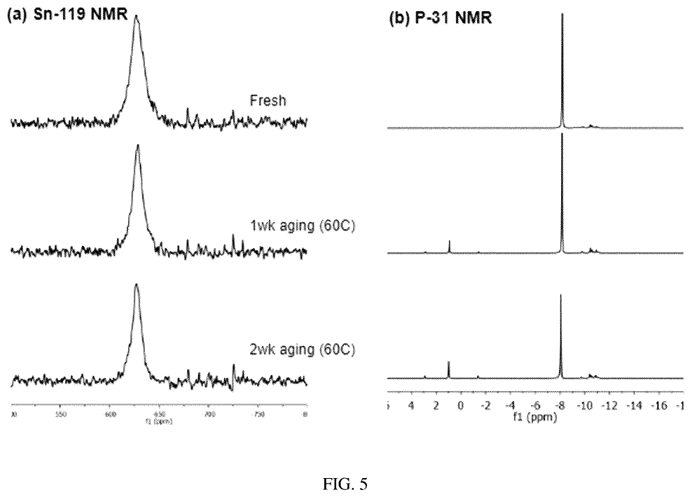

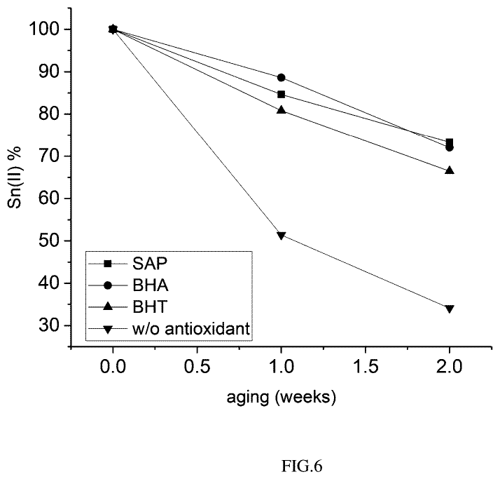

Nuclear Magnetic Resonance (NMR): The stability of Sn (II) was further examined by Nuclear Magnetic Resonance (NMR). NMR measurements were performed on samples in the presence of 10% deuterium oxide (D20). All NMR spectra were acquired on a Bruker Avance spectrometer (Bruker-Biospin, Billerica, MA, USA) with a 5 mm liquid nitrogen cryogenic probe operating at 202 MHz for ³¹P, and 186.5 MHz ¹¹⁹Sn at 25° C. ¹¹⁹Sn and ³¹P NMR spectra were acquired on the samples of SnF₂-TSPP with three antioxidants: SAP, BHA, or BHT. FIG. 3-5 show ¹¹⁹Sn and ³¹P NMR spectra of the samples upon aging. In the ¹¹⁹Sn NMR spectra, the peak positioned at around −630 ppm corresponds to Sn(II) in solution at neutral pH. Once Sn(II) was oxidized to Sn(IV) upon aging, the Sn(II) peak intensity decreased and small Sn(IV) spikes appeared between −680 and −750 ppm. The relative amounts of Sn(II) compared to fresh sample were calculated by integrating the peak corresponding to Sn(II) in ¹¹⁹Sn NMR spectra. The capacity of three oxidants to stabilize Sn(II) was compared by measuring the Sn(II) content in the samples. The result is shown in FIG. 6. About 80% of Sn(II) was left in the samples in the presence of BHA and SAP after 2 weeks of aging at 60° C. BHT sample had 70% Sn(II) remaining after 2 weeks of aging. This result shows that three antioxidants can stabilize the Sn(II), consistent with the FTIR and titration data. In the ³¹P NMR spectra, the peak positioned at −8 ppm corresponds to TSPP. The TSPP peak gradually decreased due to temperature-driven decomposition upon aging (FIG. 3-5).

### Example 2

In order to identify the optimal concentration of antioxidants (SAP and sodium ascorbate) that can increase stannous stability in aqueous solutions, samples of Sn(II) with TSPP and various concentrations of SAP or sodium ascorbate were tested for the stability of Sn(II). Samples of Sn(II) were prepared by dissolving TSPP and SnF₂ in water with the final concentration of SnF₂ equal to 2 wt %. Antioxidant (SAP or sodium ascorbate) was added to the samples with the molar ratio of SnF₂:TSPP:antioxidant equal to 1:1:x, where x was between 0.05 and 0.3. The pH of the solutions was adjusted with NaOH to pH=7.

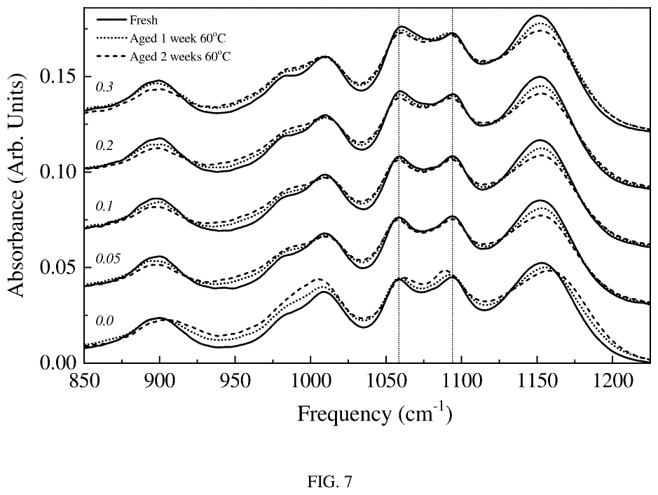

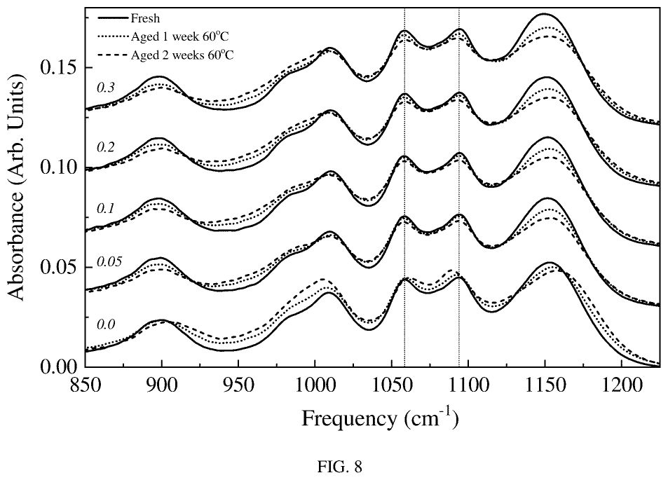

Fourier transform infrared spectroscopy (FTIR): Fourier transform infrared spectroscopy was performed as described in Example 1. The absorption spectrum of SnF₂-TSPP solution in the 850-1240 cm⁻¹ region at different time points: fresh, 1 week and 2 weeks aged at 60° ° C. was examined. Clear changes in the pyrophosphate bands of the stannous-pyrophosphate complex were observed with aging. The transformation of the complex was manifested in the peak shifts and intensity changes of phosphate bands. As an example, a doublet with peaks near 1058 and 1094 cm⁻¹ exhibited an inward band shifting towards a greater overlap of the two features after 2 weeks at 60° ° C., while the 900 and 1153 cm⁻¹ bands displayed a blue-shift with sample aging. The behavior of these absorption peaks was examined. The results are shown in FIGS. 7 and 8. The infrared data show that the improved stability of stannous-pyrophosphate can be achieved even at the very low SAP/Sn molar ratio of 0.05. The 0.05 and 0.1 SAP/Sn molar ratios showed overall smaller changes in the phosphate bands with aging compared to 0.2 and 0.3 ratios (FIG. 7). This result suggests that using a low concentration of SAP as little as 0.05 SAP/Sn molar ratio can increase the stability of stannous-pyrophosphate complex and slow oxidation of stannous. Similarly, the addition of sodium ascorbate to SnF₂-TSPP solution increased the stability of the stannous-pyrophosphate complex. Furthermore, the lower concentrations (0.05 and 0.1 ratios) of sodium ascorbate showed comparable or even slightly better effect on complex stability compared to 0.2 and 0.3 ratios (FIG. 8).

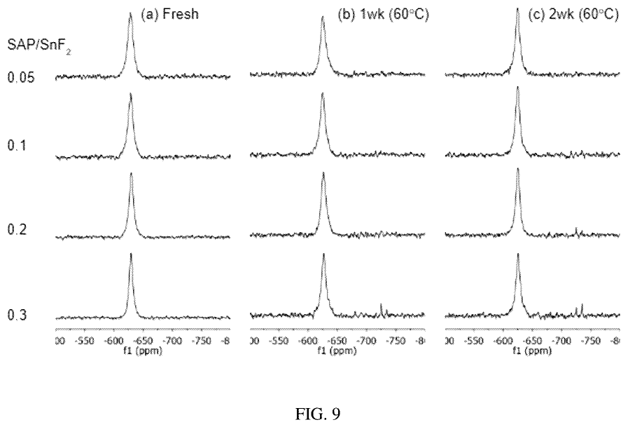

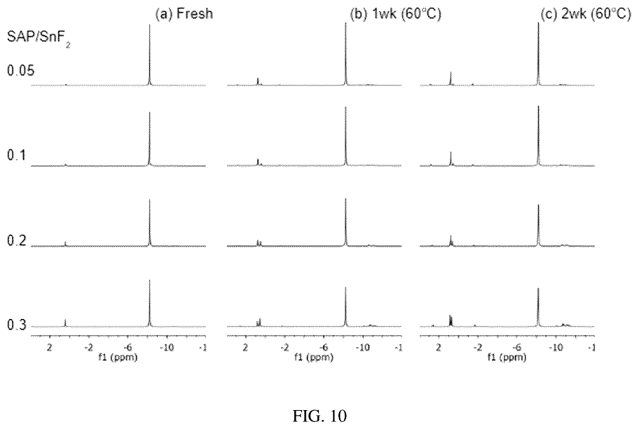

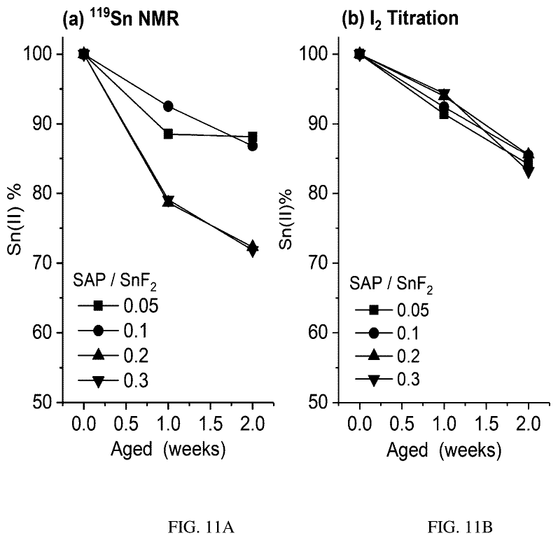

Nuclear Magnetic Resonance (NMR): The stability of Sn (II) was further examined by Nuclear Magnetic Resonance (NMR). NMR measurements were performed as described in Example 1. ¹¹⁹Sn and ³¹P NMR spectra were acquired on the samples of SnF₂-TSPP with SAP or sodium ascorbate at various SAP/Sn molar ratios, specifically between 0.05 and 0.3, upon aging at 60° C. FIGS. 9 and 10 show ¹¹⁹Sn and ³¹P NMR spectra of the samples upon aging. In the ¹¹⁹Sn NMR spectra, the peak positioned at around −630 ppm corresponds to Sn(II) in solution at neutral pH. Once Sn(II) was oxidized to Sn(IV), the Sn(II) peak intensity decreased and small Sn(IV) spikes appeared between −680 and −750 ppm. As seen in FIG. 9, the line shape and peak intensity of the ¹¹⁹Sn NMR peak corresponding to Sn(II) at around −630 ppm did not show a significant difference upon aging at 60° C. However, small Sn(IV) peaks appeared at around −730 ppm after 1 week and 2 weeks aging at SAP/SnF₂ ratio=0.3. In the ³¹P NMR spectra, the peak positioned at −8 ppm corresponds to TSPP. The TSPP peak gradually decreased due to temperature-driven decomposition upon aging (FIG. 10). For the sample at longer aging time and higher SAP/Sn ratio, the ³¹P NMR peak intensity of TSPP at −8 ppm showed a clear decrease while the orthophosphate peak at 1 ppm increased, indicating TSPP degrades to orthophosphate upon aging. The relative amounts of Sn(II) compared to fresh sample were quantified based on the Sn(II) NMR peak integral (FIG. 11A). The result shows that the Sn(II) content at lower mole ratio of SAP/Sn (0.05, 0.1) remained at about 90% after 2 weeks of aging, while Sn(II) sample with higher SAP/Sn ratio (0.2, 0.3) had only 72% Sn(II) left after the same aging period.

Iodine titration: The stability of Sn (II) was further examined by iodine titration assay. Titration measurements were performed as described in Example 1. Sn(II) % measured by I₂ titration shows all of the samples had about 85% Sn(II) % left after 2 weeks of aging (FIG. 11B).

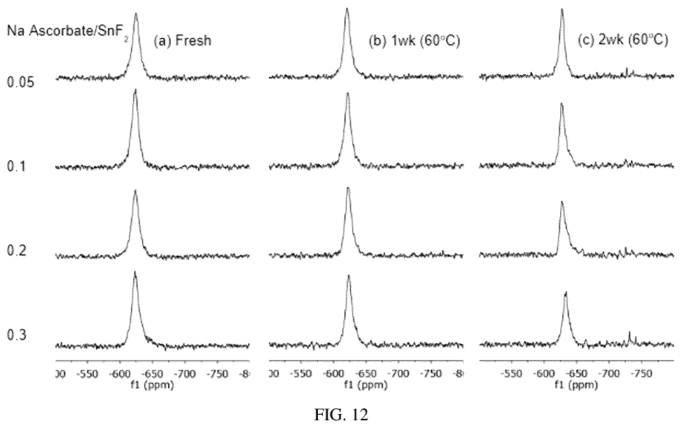

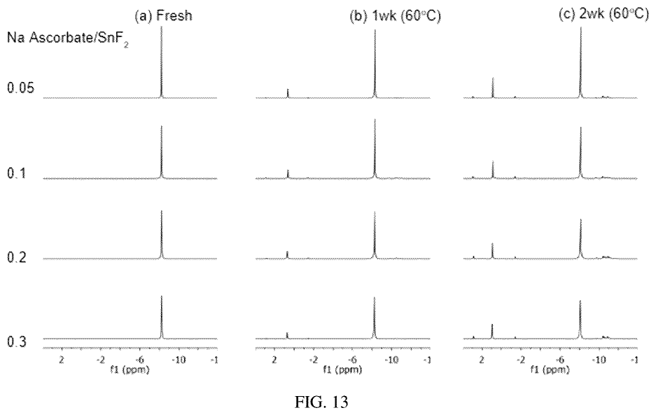

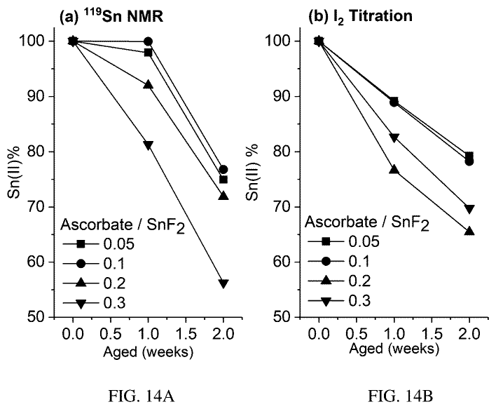

The concentration dependence of sodium ascorbate was also investigated. The results are summarized in FIG. 12-14. The results are similar to that of the SnF₂-TSPP-SAP. Sodium ascorbate at a lower mole ratio of ascorbate/Sn (0.05, 0.1) had a better capability to stabilize Sn(II). For example, according to the titration result, Sn(II) % in the sample with a lower ascorbate/Sn ratio had about 80% after 2 weeks of aging at 60° C., while Sn(II) % was 70-75% in the sample with a higher ascorbate/Sn ratio. Overall, the results show that SAP has a slightly better capability to stabilize Sn(II) than sodium ascorbate.

Head-space O₂ consumption: Stannous stability in presence of small amounts of SAP was further examined by measuring the rate of oxygen consumption during Sn(II) oxidation process. Stannous oxidation reactions were carried out in a closed 250 mL round-bottom flask and were monitored by pressure changes in the gaseous (air) headspace above the solution at a constant temperature of 25±0.5° C. In a typical experiment the vessel was filled with 100 mL of solution containing 29 mM SnF₂ and 29 mM TSPP, with or without SAP. SnF₂ was added last as a powder, and the flask was immediately scaled. The solution was stirred magnetically at 750 rpm and the differential pressure was recorded over about 6.5 hour period. Digital manometer (APT Instruments, MP2000) connected to the flask was used to record the differential pressure. The pH of the solution was about 6.5 and did not exhibit significant changes during the reaction.

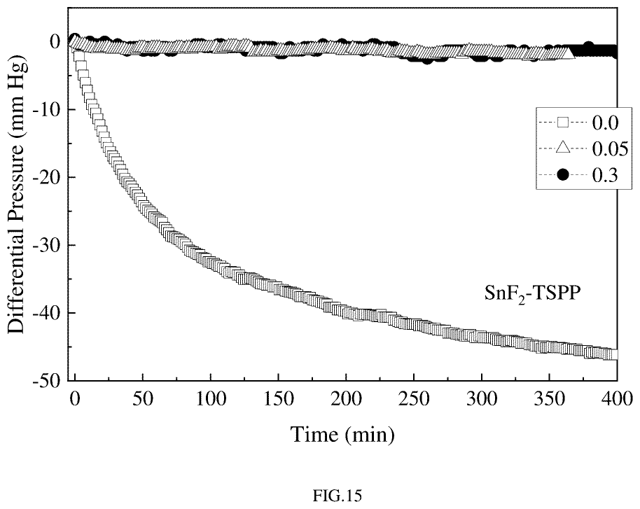

The progress of the stannous oxidation reaction was monitored through gas-phase pressure changes above the solution of interest reflecting the consumption of oxygen during the reaction. FIG. 15 shows the differential pressure readings as a function of time for solutions containing SnF₂ and TSPP with or without SAP. In the absence of SAP, a continuous drop in pressure was observed during about 6.5 hours of data collection, indicating a rapid consumption of O₂ due to stannous oxidation process. In contrast, when SAP (two examples of SAP/Sn molar ratios of 0.05 and 0.3 are shown) is present in the same solution, no significant pressure drop was observed over the same period of time, confirming the suppressing effect of SAP on the oxidation kinetics of stannous. Both concentrations of SAP (i.e., 0.05 and 0.3 SAP/Sn molar ratios) showed similar effect on stannous stability within the time frame of the experiment.

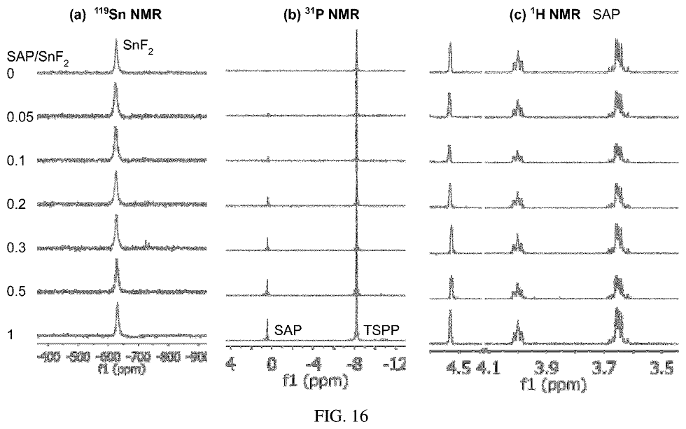

NMR: NMR measurements were carried out on samples prepared from SnF₂, TSPP and SAP at 1:1:x mole ratio, where x was between 0 and 1. The final SnF₂ concentration in solution was 2 wt %. The pH of the solution was adjusted with NaOH and HCl to pH 7. NMR measurements were performed as described in Example 1 with a 5 mm liquid nitrogen cryogenic probe operating at 500 MHz for ¹H. The results are shown in FIG. 16A-C. Sn(II) chemical shift of SnF₂ is at 630 ppm (FIG. 16A). Phosphorus peak of SAP is at 0.6 ppm and the phosphate peak of TSPP is at −8.2 ppm (FIG. 16B). FIG. 16C represents the proton peaks originating from SAP. As can be seen in FIG. 16A-C, the ³¹P/¹H chemical shift of SAP and ¹¹⁹Sn chemical shift are independent of the various SAP concentrations, suggesting that SAP and SnF₂ do not interact with each other in the presence of TSPP.

### Example 3

To investigate the effect of SAP on the stability of Sn(II) in toothpaste formulation, SnF₂ and various concentrations of SAP (from 0-1%) were post-added to a base formulation and speed mixed until they became homogeneous. The formula of the base formulation is shown in Table 2 and the amounts of materials used in the preparation of tested toothpastes are shown in Table 3.

TABLE 2

| | Amount |
|---|---|
| Ingredient | (g) |
| Polyethylene Glycol | 20.07 |
| Xanthan Gum | 4.02 |
| Sorbitol - Non-Crystal - (70% Soln) | 620.9 |
| Silica | 150.87 |
| Synthetic Thickening Silica | 80.23 |
| Sodium Lauryl Sulfate | 36.73 |
| Water | 41.30 |
| Sodium Saccharin | 4.04 |
| Tetrasodium pyrophosphate | 12.34 |
| Cocamidopropyl betaine | 12.5 |

TABLE 3

| | TP Placebo | | |
|---|---|---|---|
| Sample Name | Base (g) | SnF₂ (g) | SAP (g) |
| 1% SAP | 98.01 | 0.46 | 1.00 |
| 0.75% SAP | 98.06 | 0.45 | 0.75 |
| 0.5% SAP | 98.05 | 0.45 | 0.50 |
| 0.25% SAP | 98.04 | 0.45 | 0.25 |
| 0.1% SAP | 98.05 | 0.45 | 0.10 |
| 0.05% SAP | 98.05 | 0.45 | 0.05 |
| Placebo 0% SAP | 98.04 | 0.46 | — |
| Placebo 0% SnF₂ | 98.09 | — | 1.00 |

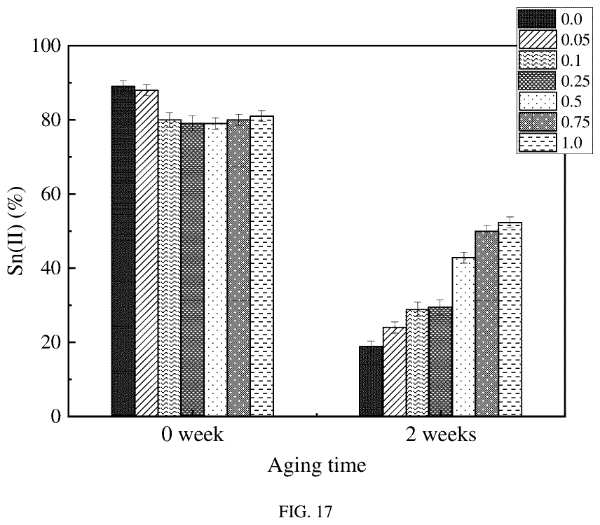

As shown in Table 2, the base formulation was prepared without SnF₂, flavor, or dyes. The toothpaste formulations (Table 3) were transferred to toothpaste tubes and sealed. The tubes were aged in the oven at 60° C. for 2 weeks. The toothpastes were analyzed by titration for Sn(II) present before and after aging in the oven at 60° C. Titration measurements were performed as described in Example 1. The result is shown in FIG. 17. As shown in FIG. 17, as little as 0.05% SAP slowed down oxidation of Sn(II) in the toothpaste. As concentration of SAP increased, the stability of Sn(II) also increased. With 1% SAP, the highest concentration tested, 52% of Sn(II) remained, showing a considerable improvement compared to the control with no SAP which had 19% Sn(II) remaining after the 2 week aging period. This result shows that a low concentration of SAP as little as 0.05% starts to slow down the oxidation of Sn(II) in toothpaste formulation.

A control toothpaste with 1% SAP but no SnF₂ was prepared and titrated under the same conditions. The control toothpaste showed no interference of SAP or other toothpaste ingredients with the titration method during the 2 weeks of aging.

### Example 4

SAP and ascorbic acid were separately formulated at 0.5 wt. % or 1 wt. % in a common toothpaste formulation containing 0.454 wt. % stannous fluoride and 1 wt. % zinc phosphate, where the weight percentages are based on the total weight of the oral care composition. The formula of the base formulation are shown in Table 4.

TABLE 4

| US INCI | Formula | Formula | Formula | Formula | Formula |
|---|---|---|---|---|---|
| Name | A (wt. %) | B (wt. %) | C (wt. %) | D (wt. %) | E (wt. %) |
| PEG 600 | 2 | 2 | 2 | 2 | 2 |
| Flavoring | 0.41 | 0.41 | 0.41 | 0.41 | 0.41 |
| agent | | | | | |
| Stannous | 0.454 | 0.454 | 0.454 | 0.454 | 0.454 |
| Fluoride | | | | | |
| TSPP | 2 | 2 | 2 | 2 | 2 |
| Sorbitol | 40.2 | 39.7 | 39.2 | 39.7 | 39.2 |
| Water | 18.3 | 18.3 | 18.3 | 18.3 | 18.3 |
| Hydrated | 21.5 | 21.5 | 21.5 | 21.5 | 21.5 |
| Silica | | | | | |
| Glycerin | 6 | 6 | 6 | 6 | 6 |
| Flavor | 1.63 | 1.63 | 1.63 | 1.63 | 1.63 |
| Surfactant | 2.75 | 2.75 | 2.75 | 2.75 | 2.75 |
| Thickeners | 2.1 | 2.1 | 2.1 | 2.1 | 2.1 |
| Trisodidum | 1 | 1 | 1 | 1 | 1 |
| Citrate | | | | | |
| Citric Acid | 0.2 | 0.2 | 0.2 | 0.2 | 0.2 |
| Zinc | 1 | 1 | 1 | 1 | 1 |
| Phosphate | | | | | |
| Colorant | 0.5 | 0.5 | 0.5 | 0.5 | 0.5 |
| Ascorbic | 0 | 0.5 | 1.0 | 0 | 0 |
| Acid | | | | | |
| Ascorbyl | 0 | 0 | 0 | 0.5 | 1.0 |
| Phosphate | | | | | |

The toothpastes were placed in controlled temperature and humidity chambers (40° C./75% RH or 30° C./65% RH) for a specified period of time. The Sn(II) concentration was measured at different time points: fresh, 4 weeks, 8 weeks, and 13 weeks. The results are shown in Table 5. Sn(II) results show that SAP is effective in maintaining active tin and 1 wt. % SAP (Formulations D and E) provided better stability over a common formula without SAP. In contrast, formulations with ascorbic acid (Formulations B and C) did not stabilize Sn(II).

TABLE 5

| | | Formula | Formula | Formula | Formula | Formula |
|---|---|---|---|---|---|---|
| | | A | B | C | D | E |
| Total Tin | Initial | 0.34 | 0.34 | 0.33 | 0.33 | 0.35 |
| (mol %) | | | | | | |
| Sol. Tin | Initial | 0.25 | 0.34 | 0.33 | 0.25 | 0.34 |
| (mol %) | 4 wks- | 0.24 | 0.29 | 0.29 | 0.22 | 0.23 |
| | 40° C./ | | | | | |
| | 75% RH | | | | | |
| | 8 wks- | 0.22 | 0.2 | 0.22 | 0.22 | 0.23 |
| | 40° C./ | | | | | |
| | 75% RH | | | | | |
| | 13 wks- | 0.23 | 0.18 | 0.22 | 0.23 | 0.23 |
| | 30° C./ | | | | | |
| | 65% RH | | | | | |
| | 13 wks- | 0.19 | 0.15 | 0.15 | 0.19 | 0.22 |
| | 40° C./ | | | | | |
| | 75% RH | | | | | |
| Sn (II) | Initial | 0.24 | 0.22 | 0.22 | 0.27 | 0.32 |
| (mol %) | 4 wks- | 0.19 | 0.18 | 0.2 | 0.21 | 0.21 |
| | 40° C./ | | | | | |
| | 75% RH | | | | | |
| | 8 wks- | 0.19 | 0.13 | 0.17 | 0.19 | 0.21 |
| | 40° C./ | | | | | |
| | 75% RH | | | | | |
| | 13 wks- | 0.21 | 0.16 | 0.15 | 0.19 | 0.22 |
| | 30° C./ | | | | | |
| | 65% RH | | | | | |
| | 13 wks- | 0.17 | 0.1 | 0.1 | 0.18 | 0.21 |
| | 40° C./ | | | | | |
| | 75% RH | | | | | |

1. An oral care composition comprising: a stannous ion source, tetrasodium pyrophosphate and an antioxidant selected from ascorbyl phosphate, ascorbate, butylated hydroxytoluene (BHT), and butylated hydroxyanisole (BHA).

2. The oral care composition of claim 1, wherein the antioxidant is ascorbyl phosphate.

3. The oral care composition of claim 2, wherein the ascorbyl phosphate is sodium ascorbyl phosphate.

4. The oral care composition of claim 1, wherein the molar ratio of stannous:TSPP:antioxidant is 1:0.5-1.5:0.05-1.

5. The oral care composition of claim 4, wherein the oral care composition has a molar ratio of stannous to TSPP to antioxidant is 1:0.5-1.5:0.05-0.3.

6. The oral care composition of claim 1, wherein the stannous ion source is selected from the group consisting of stannous fluoride, stannous gluconate, stannous phosphate, stannous pyrophosphate, stannous acetate, stannous sulfate, stannous chloride, and a combination thereof.

7. The oral care composition of claim 1, wherein the stannous ion source is present in an amount of from about 0.1 to about 5%, by weight based on the total weight of the oral care composition.

8. The oral care composition of claim 1, wherein the composition comprises a fluoride ion source.

9. The oral care composition of claim 1, wherein the oral care composition comprises a zinc ion source.

10. The oral care composition of claim 1, wherein the oral care composition comprises a basic amino acid.

11. The oral care composition of claim 1, wherein pH of the composition is from about 6.5 to about 7.5.

12. The oral care composition of claim 11, wherein pH of the oral care composition is from 6.6 to 7.4.

13. The oral care composition of claim 1, wherein the oral care composition does not contain potassium nitrate.

14. The oral care composition of claim 1, wherein the oral care composition is a dentifrice, a toothpaste, a gel, or a mouthwash.

15. (canceled)

16. The oral care composition of claim 1, wherein the oral care composition has a weight ratio of the total amount of antioxidant to the total amount of stannous ion source and TSPP of about 1:1.5 to about 1:8.

17. A method of (i) reducing or inhibiting formation of dental caries, (ii) reducing, repairing or inhibiting pre-carious lesions of the enamel, (iii) reducing or inhibiting demineralization and promoting remineralization of the teeth, (iv) reducing hypersensitivity of the teeth, (v) reducing or inhibiting gingivitis, (vi) promoting healing of sores or cuts in the oral cavity, (vii) reducing levels of acid producing bacteria, (viii) reducing or inhibiting microbial biofilm formation in the oral cavity, (ix) reducing or inhibiting plaque formation in the oral cavity, (x) promoting systemic health, or (xi) cleaning teeth and oral cavity, comprising applying an oral care composition according to claim 1 to the oral cavity.

18. (canceled)
